(19) United States
(12) Patent Application Publication
(10) Pub. No.: US 2013/0189157 A1
(43) Pub. Date: Jul. 25, 2013

(54) ROTATING DEVICE AND BLOOD ANALYSIS EQUIPMENT

(71) Applicant: Ta-Hsiang Wang, Hsinchu (TW)

(72) Inventor: Ta-Hsiang Wang, Hsinchu (TW)

(73) Assignee: LITE-ON IT CORPORATION, TAIPEI CITY (TW)

(21) Appl. No.: 13/414,729

(22) Filed: Mar. 8, 2012

(30) Foreign Application Priority Data
Jan. 20, 2012 (CN) 201210017855.6

(51) Int. Cl. G01N 33/50; B01L 3/00

(52) U.S. Cl. 422/68.1; 422/547

(57) ABSTRACT

A rotating device including a main body, a tray, a cover and a motor is provided. The tray is disposed in the main body and is adapted to support an object. The cover is disposed in the main body and is adapted to cover the object. The motor is disposed in the main body and is adapted to drive the object to rotate. When the object rotates to cause a liquid on the object to move away from the object, the cover prevents the liquid from moving to the main body. In addition, a blood analysis equipment is also provided.

### CROSS-REFERENCE TO RELATED APPLICATION

This application claims the priority benefit of China application serial no. 201210017855.6, filed on Jan. 20, 2012. The entirety of the above-mentioned patent application is hereby incorporated by reference herein and made a part of this specification.

### BACKGROUND OF THE INVENTION

1. Field of the Invention

The invention relates to an analysis equipment and a rotating device thereof, and more particularly to a blood analysis equipment and a rotating device thereof.

2. Description of Related Art

In clinical examinations of blood components, various types of blood analysis apparatuses may be used to analyze blood. For example, a blood analysis apparatus of prior art is designed to be able to divide whole blood into blood serum or plasma and components containing blood cells and to analyze the separated blood plasma. A method of separating the blood plasma from the whole blood sample includes: disposing the whole blood sample in a blood analysis cassette, and then rotating the blood analysis cassette to separate the plasma of a lower specific gravity from the whole blood sample with centrifugation force.

Based on the above method, the blood plasma may be analyzed to obtain required parameters. For patients under complicated medical treatment procedures such as operations and patients under special care for serious diseases, it is especially important to determine the parameters of the patients. The parameters provide medical staff with important information regarding statuses of the patients and allow the medical staff to provide proper and complete treatments to the patients. However, in the above process of separating the blood plasma from the whole blood sample, if the medical staff fail to perform proper cleaning in advance, then in operating the blood analysis cassette, other liquid (such as potion or other blood) often attach to the blood analysis cassette. When the blood analysis cassette rotates, the liquid attached to the blood analysis cassette is often tossed to other elements inside the blood analysis apparatus and the blood analysis equipment becomes moldy or bacteria grow therein, which causes the properties of the blood analysis apparatus to decline and influences the analysis accuracy.

### SUMMARY OF THE INVENTION

The invention provides a rotating device; a blood analysis equipment using the rotating device has a better analysis accuracy.

The invention provides a blood analysis equipment has a better analysis accuracy.

The invention proposes a rotating device including a main body, a tray, a cover and a motor. The tray is disposed in the main body and is adapted to support an object. The cover is disposed in the main body and is adapted to cover the object. The motor is disposed in the main body and is adapted to drive the object to rotate. When the object rotates to cause a liquid on the object to move away from the object, the cover prevents the liquid from moving to the main body.

In an embodiment of the invention, the tray has a projection and a groove adjacent to each other. The projection is adapted to support the object. The liquid rotating with the object moves to the cover, and flows to the groove from the cover.

In an embodiment of the invention, the cover includes a top wall and at least a side wall. The side wall is connected to a side of the top wall, wherein the side wall is adapted to guide the liquid to flow to the groove.

In an embodiment of the invention, a bottom of the side wall has a guiding plate, and the guiding plate tilts from the bottom of the side wall towards the groove.

In an embodiment of the invention, a bottom of the side wall has a guiding plate, and the guiding plate is parallel to the top wall.

In an embodiment of the invention, the tray has a positioning slot. The object has a protrusion portion adapted to insert into the positioning slot to position the object on the tray.

In an embodiment of the invention, the positioning slot passes through the tray, and the motor is located under the tray and connected to the object through the positioning slot.

In an embodiment of the invention, the tray is slidably disposed in the main body and suitable for sliding relative to the main body to move into or out of the main body. When the tray moves into the main body, the object supported on the tray is aligned to the cover.

In an embodiment of the invention, the cover is detachably assembled in the main body.

The invention proposes a blood analysis equipment including a rotating device and an analysis unit. The rotating device includes a main body, a tray, a cover and a motor. The tray is disposed in the main body and is adapted to support an object, wherein the object is adapted to contain a tested blood. The cover is disposed in the main body and is adapted to cover the object. The motor is disposed in the main body and is adapted to drive the object to rotate for separating an ingredient from the tested blood. When the object rotates to cause a liquid on the object to move away from the object, the cover prevents the liquid from moving to the main body. The analysis unit is for analyzing the ingredient.

In an embodiment of the invention, the component is blood plasma. The object is further adapted to contain a reagent. The motor is adapted to drive the tray to rotate so as to mix the blood plasma and the reagent.

In an embodiment of the invention, the tray has a projection and a groove adjacent to each other. The projection is adapted to support the object. The liquid moves to the cover, and flows to the groove from the cover.

In an embodiment of the invention, the cover includes a top wall and at least a side wall. The side wall is connected to a side of the top wall, wherein the side wall is adapted to guide the liquid to flow to the groove.

In an embodiment of the invention, a bottom of the side wall has a guiding plate. The guiding plate tilts from the bottom of the side wall towards the groove.

In an embodiment of the invention, a bottom of the side wall has a guiding plate. The guiding plate is parallel to the top wall.

In an embodiment of the invention, the tray has a positioning slot. The object has a protrusion portion adapted to insert into the positioning slot to position the object on the tray.

In an embodiment of the invention, the positioning slot passes through the tray. The motor is located under the tray and connected to the object through the positioning slot.

In an embodiment of the invention, the tray is slidably disposed in the main body and suitable for sliding relative to the main body to move into or out of the main body. When the tray moves into the main body, the object supported on the tray is aligned to the cover.

In an embodiment of the invention, the cover is detachably assembled in the main body.

Based on the above, in the blood analysis equipment of the invention, the object for containing the tested blood is supported by the tray of the rotating device, and the cover covers the object. Therefore, when the motor drives the object to rotate, the cover prevents the liquid (such as medicinal liquid or other blood) adhered to the object from being tossed to other elements in the main body due to the centrifugation force, which ensures that the inside of the main body is clean, prevents the blood analysis equipment from becoming moldy or bacteria from growing, and makes the blood analysis equipment have a better analysis accuracy.

In order to make the aforementioned features and advantages of the invention more comprehensible, embodiments accompanying figures are described in detail below.

### BRIEF DESCRIPTION OF THE DRAWINGS

The accompanying drawings constituting a part of this specification are incorporated herein to provide a further understanding of the invention. Here, the drawings illustrate embodiments of the invention and, together with the description, serve to explain the principles of the invention.

FIG. 1 is a block diagram of a blood analysis equipment according to an embodiment of the invention.

FIG. 2 is a front view of the rotating device of FIG. 1.

FIG. 3 is a front view of a blood analysis equipment according to another embodiment of the invention.

FIG. 4 is a front view of a blood analysis equipment according to another embodiment of the invention.

FIG. 5 is a side view of the rotating device of FIG. 2.

FIG. 6 is a side view of the tray of FIG. 2 moving out of the main body.

### DESCRIPTION OF EMBODIMENTS

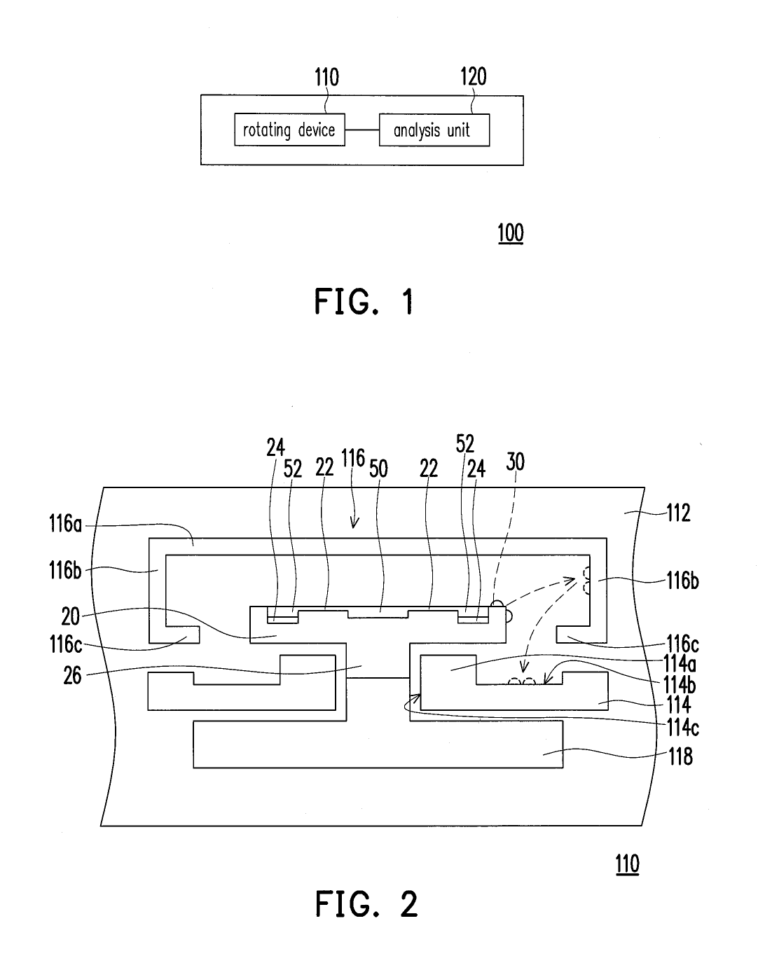

FIG. 1 is a block diagram of a blood analysis equipment according to an embodiment of the invention. FIG. 2 is a front view of the rotating device of FIG. 1. Referring to FIG. 1 and FIG. 2, in the present embodiment, a blood analysis equipment 100 includes a rotating device 110 and an analysis unit 120. The rotating device 110 includes a main body 112, a tray 114, a cover 116 and a motor 118. The tray 114 is disposed in the main body 112 and is adapted to support an object 20. The object 20 is, for example, a blood analysis cassette for containing a tested blood 50. The cover 116 is disposed in the main body 112 and is adapted to cover the upper and side areas of the object 20.

The motor 118 is disposed in the main body 112 and is adapted to drive the object 20 to rotate for separating an ingredient 52 from the tested blood 50, and the analysis unit 120 is adapted to analyze the ingredient 52. More specifically, when the motor 118 drives the object 20 to rotate, the ingredient 52 in the tested blood 50, with the help of centrifugation force, flows from a central part of the object 20 to an edge part of the object 20 through a channel 22. In FIG. 2, the channel 22 is illustrated as a straight path; however, the channel 22 may actually be a complicated winding path to properly separate the ingredient 52, which is not limited in the invention.

When the motor 118 drives the object 20 to rotate, the cover 116 prevents the liquid 30 (such as medicinal liquid or other blood) adhered to the object 20 from being tossed to the main body 112 due to the centrifugation force, which ensures that the inside of the main body 112 is clean, prevents the liquid 30 from adhering to other elements in the blood analysis equipment 100 and causing the elements to be moldy or bacteria to grow, and makes the blood analysis equipment 100 have a better analysis accuracy.

In the present embodiment, the ingredient 52 separated from the tested blood 50 is, for example, blood plasma, and the object 20 is further adapted to contain a reagent 24. When the motor 118 drives the tray 114 to rotate and makes the ingredient 52 to be separated to flow to the edge part of the object 20, the ingredient 52 mixes with the reagent 24 so as to facilitate an analysis.

In the present embodiment, the tray 114 has a projection 114a, a groove 114b, and a positioning slot 114c. The projection 114a is adapted to support the object 20 to move into and out of the main body 112. The positioning slot 114c passes through the tray 114, and the object 20 has a protrusion portion 26 which is adapted to insert into the positioning slot 114c to position the object 20 on the tray 114. The motor 118 is located under the tray 114 and connected to the object 20 through the positioning slot 114c. When the motor 118 drives the tray 114 to rotate, the liquid 30 is tossed to the cover 116 with the help of centrifugation force and flows from the cover 116 to the groove 114b (as indicated by the dashed arrow).

More specifically, the cover 116 includes a top wall 116a and two side walls 116b. The two side walls 116b are connected to two sides of the top wall 116a respectively; a bottom of each of the side walls 116b has a guiding plate 116c which is parallel to the top wall 116a. In this way, the liquid 30 tossed off the object 20 to the cover 116 may flow to the groove 114b through the guidance of each of the side walls 116b and their guiding plates 116c. In addition, the cover 116 of the present embodiment is assembled in the main body 112 in, for example, a detachable way, such as being disposed on a rail or fixed with hooks. When the cover 116 is to be detached, the hooks are pushed to release the cover 116, or the cover 116 slides out along the rail, which facilitates the installment, detachment and cleaning of the cover 116.

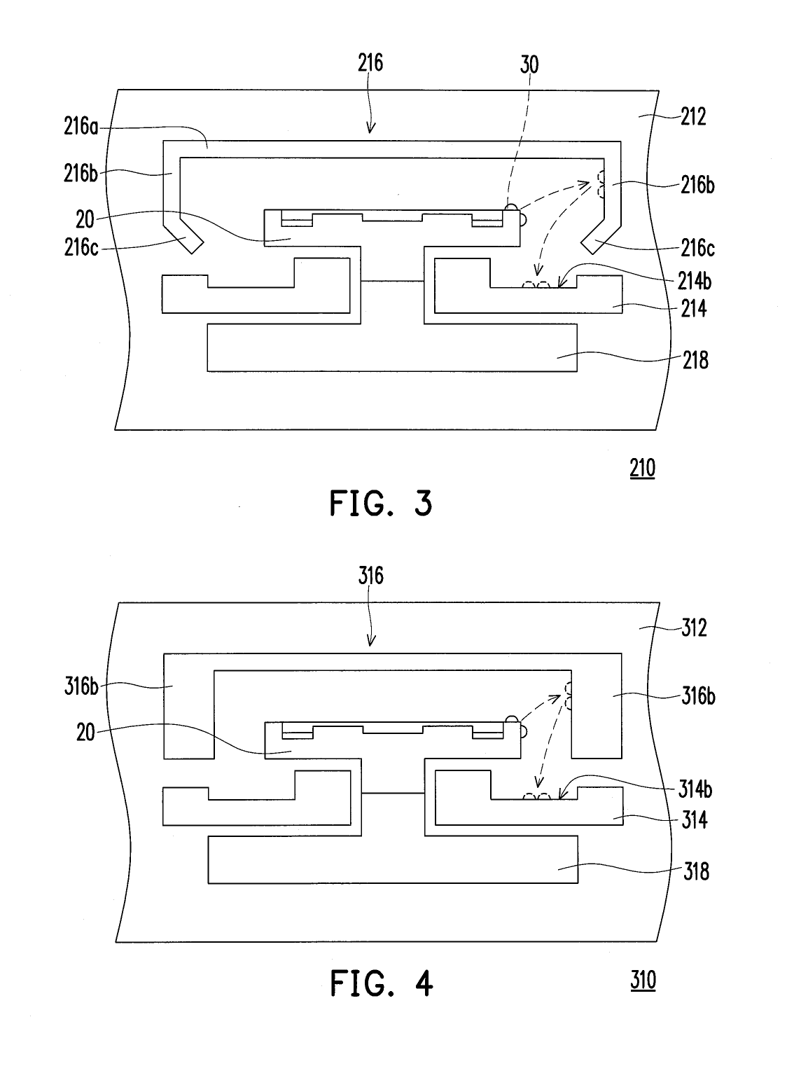

The invention does not limit shapes and sizes of the side walls of the cover. Examples with reference to the drawings are provided below to further illustrate it. FIG. 3 is a front view of the rotating device according to another embodiment of the invention. Referring to FIG. 3. In the rotating device 210 of the present embodiment, the main body 212, the tray 214, the cover 216 and the motor 218 are disposed in a way similar to that of the main body 112, the tray 114, the cover 116 and the motor 118 in FIG. 2. Therefore, their disposition will not be described in detail herein. The rotating device 210 of the present embodiment differs from the rotating device 110 of FIG. 2 in that the guiding plate 216c does not parallel the top wall 216a of the cover 216 but tilts from the bottom of the side wall 216a towards the groove 214b. In this way, it is easier for the liquid 30 tossed off the object 20 to the cover 216 to flow to the groove 214b along the guiding plate 216c.

FIG. 4 is a front view of the rotating device according to another embodiment of the invention. In the rotating device 310 illustrated in FIG. 4, the main body 312, the tray 314, the cover 316 and the motor 318 are disposed in a way similar to that of the main body 112, the tray 114, the cover 116 and the motor 118 in FIG. 2. Therefore, their disposition will not be described in detail herein. The rotating device 310 of the present embodiment differs from the rotating device 110 of FIG. 2 in that at the bottom of the side wall 316b, there is not a guiding plate 116c as shown in FIG. 2. The size of the top wall and the location of the side wall 316b of the cover 316 are properly arranged so that with the vertical side wall 316b, the liquid 30 tossed off the object 20 to the cover 316 directly flows to the groove 314b along the side wall 316b.

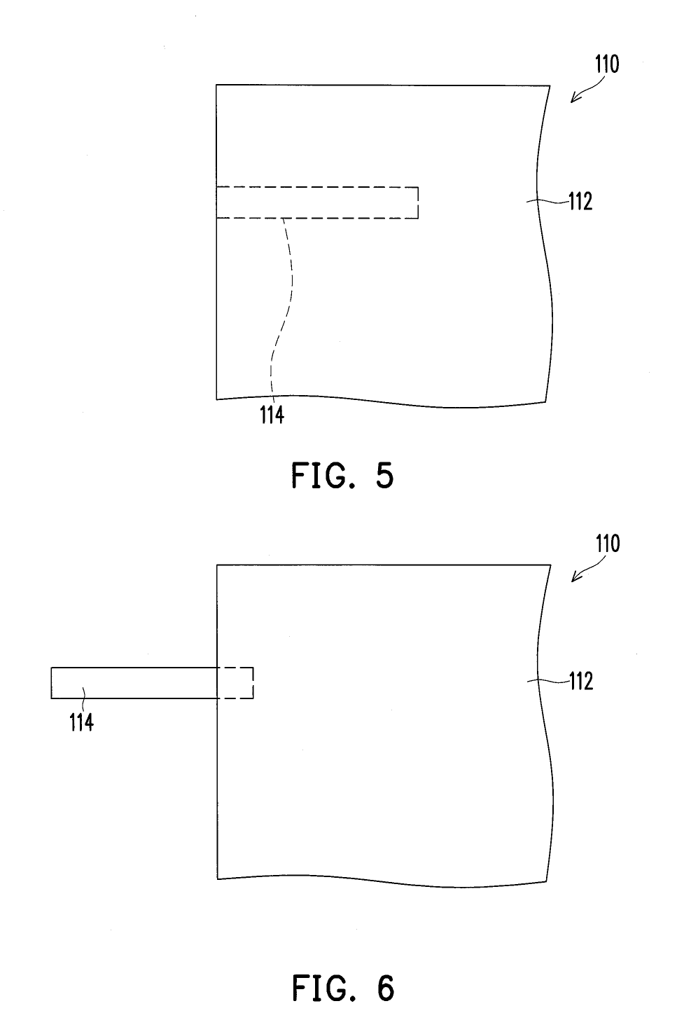

FIG. 5 is a side view of the rotating device of FIG. 2. FIG. 6 is a side view of the tray of FIG. 2 moving out of the main body. Referring to FIG. 5 and FIG. 6. In the present embodiment, the tray 114 is slidably disposed in the main body 112 and suitable for sliding relative to the main body 112 to move out of the main body 112 (as shown in FIG. 6) or into the main body 112 (as shown in FIG. 5). When the tray 114 moves into the main body 112, the object 20 supported by the tray 114 is aligned to the cover 116 as shown in FIG. 2. When the liquid 30 adhered to the object 20 is tossed off, it is obstructed by the cover 116 and falls on the tray 114. When the tray 114 moves out of the main body 112, it is convenient for users to clean the collected liquid 30 on the groove 114b of the tray 114.

Based on the above, in the blood analysis equipment of the invention, the object for containing the tested blood is supported by the tray of the rotating device, and the cover covers the object. Therefore, when the motor drives the object to rotate, the cover prevents the liquid (such as medicinal liquid or other blood) adhered to the object from being tossed to other elements in the main body due to the centrifugation force, which ensures that the inside of the main body is clean, prevents the blood analysis equipment from becoming moldy or bacteria from growing, and makes the blood analysis equipment have a better analysis accuracy.

Though the invention has been disclosed above by the embodiments, they are not intended to limit the invention. It will be apparent to one of ordinary skill in the art that modifications and variations to the described embodiments may be made without departing from the spirit and scope of the invention. Therefore, the protecting range of the invention falls in the appended claims.

What is claimed is:

1. A rotating device, comprising:
a main body;
a tray disposed in the main body and adapted to support an object;
a cover disposed in the main body and adapted to cover the object; and
a motor disposed in the main body and adapted to drive the object to rotate, wherein when the object rotates to cause a liquid on the object to move away from the object, the cover prevents the liquid from moving to the main body.

2. The rotating device according to claim 1, wherein the tray has a projection and a groove adjacent to each other, the projection is adapted to support the object, the liquid rotating with the object moves to the cover and flows to the groove from the cover.

3. The rotating device according to claim 2, wherein the cover comprises:
a top wall;
at least a side wall connected to a side of the top wall, wherein the side wall is adapted to guide the liquid to flow to the groove.

4. The rotating device according to claim 3, wherein a bottom of the side wall has a guiding plate, and the guiding plate tilts from the bottom of the side wall towards the groove.

5. The rotating device according to claim 3, wherein a bottom of the side wall has a guiding plate, and the guiding plate is parallel to the top wall.

6. The rotating device according to claim 1, wherein the tray has a positioning slot, and the object has a protrusion portion adapted to insert into the positioning slot to position the object on the tray.

7. The rotating device according to claim 6, wherein the positioning slot passes through the tray, and the motor is located under the tray and connected to the object through the positioning slot.

8. The rotating device according to claim 1, wherein the tray is slidably disposed in the main body and suitable for sliding relative to the main body to move into or out of the main body, and when the tray moves into the main body, the object supported on the tray is aligned to the cover.

9. The rotating device according to claim 1, wherein the cover is detachably assembled in the main body.

10. A blood analysis equipment, comprising:
a rotating device, comprising:
a main body;
a tray disposed in the main body and adapted to support an object, wherein the object is adapted to contain a tested blood;
a cover disposed in the main body and adapted to cover the object; and
a motor disposed in the main body and adapted to drive the object to rotate for separating an ingredient from the tested blood, wherein when the object rotates to cause a liquid on the object to move away from the object, the cover prevents the liquid from moving to the main body; and
an analysis unit adapted to analyze the ingredient.

11. The blood analysis equipment according to claim 10, wherein the ingredient is blood plasma, the object is further adapted to contain a reagent, and the motor is adapted to drive the tray to rotate so as to mix the blood plasma and the reagent.

12. The blood analysis equipment according to claim 10, wherein the tray has a projection and a groove adjacent to each other, the projection is adapted to support the object, the liquid rotates with the object to move to the cover and flows to the groove from the cover.

13. The blood analysis equipment according to claim 12, wherein the cover comprises:
a top wall;
at least a side wall connected to a side of the top wall, wherein the side wall is adapted to guide the liquid to flow to the groove.

14. The blood analysis equipment according to claim 13, wherein a bottom of the side wall has a guiding plate, and the guiding plate tilts from the bottom of the side wall towards the groove.

15. The blood analysis equipment according to claim 13, wherein a bottom of the side wall has a guiding plate, and the guiding plate is parallel to the top wall.

16. The blood analysis equipment according to claim 10, wherein the tray has a positioning slot, and the object has a protrusion portion adapted to insert into the positioning slot to position the object on the tray.

17. The blood analysis equipment according to claim 16, wherein the positioning slot passes through the tray, and the motor is located under the tray and connected to the object through the positioning slot.

18. The blood analysis equipment according to claim 10, wherein the tray is slidably disposed in the main body and suitable for sliding relative to the main body to move into or out of the main body, and when the tray moves into the main body, the object supported on the tray is aligned to the cover.

19. The blood analysis equipment according to claim 10, wherein the cover is detachably assembled in the main body.
